(19) United States
(12) Patent Application Publication
(10) Pub. No.: US 2022/0011256 A1
(43) Pub. Date: Jan. 13, 2022

(54) Electrochemical Gas Sensing

(71) Applicant: Aeroqual Ltd., Auckland (NZ)

(72) Inventors: Geoffrey Stephen Henshaw, Auckland (NZ); Anna Kate Farquhar, Auckland (NZ)

(21) Appl. No.: 17/371,092

(22) Filed: Jul. 8, 2021

Related U.S. Application Data
(60) Provisional application No. 63/049,164, filed on Jul. 8, 2020.

(51) Int. Cl. G01N 27/40; G01N 33/00

(52) U.S. Cl. CPC G01N 27/40; G01N 33/0006

(57) ABSTRACT

Electrochemical gas sensors are positioned in housings having open inlets for ambient gases. Leak tight caps fit over gas inlet while currents are detected until output currents are stabilized and zero baseline currents or establish for sensor calibration. The leak tight caps are removed and replaced by caps holding porous fabric membranes over the inlets. The porous fabric membranes are made of natural fibres based on keratin, cellulose, linen, as well as man-made viscose and blends. The porous fabric membranes reduce rapid humidity responses without appreciably affecting sensor responses to target gases. The porous fabric membranes release heat when water is absorbed and absorb heat when water is released. The porous fabric membranes buffer changes in temperature and humidity without significantly decreasing the gas being detected.

This application claims the benefit of U.S. Provisional Application No. 63/049,164 filed Jul. 8, 2020, which is hereby incorporated by reference in its entirety as if fully set forth herein.

### BACKGROUND OF THE INVENTION

Electrochemical gas sensors are used to measure the concentration of electroactive gases, including NO₂ and ozone. Electrochemical gas sensors typically consist of two, three or four electrodes, surrounded by an electrolyte, encased in a housing. A typical electrochemical sensor is described in U.S. Pat. No. 6,746,587. The electroactive gas diffuses through the membrane to the working electrode, where it is either oxidised or reduced, generating an electrical current. The sensor is designed to ensure this current is diffusion limited, and therefore a linear relationship exists between concentration and current. Electrochemical gas sensors can be used to measure the concentration of gases in outdoor air. However, since gas concentrations are generally in the ppb range, the output currents are also small and can be of the same magnitude as currents generated by interferences such as fluctuations in meteorological conditions. Previous work has highlighted that rapid changes in meteorological conditions including humidity, temperature, and pressure cause transient baseline current spikes, which manifest as noise in the output signal of the gas sensor, and can mask or complicate the actual gas signal.

Methods to modify sensor design for improved measurements exist. Many of these compensate for, or prevent the sensors response to temperature, humidity, or pressure. U.S. Pat. No. 7,651,597 describes adding a second working electrode (often called the auxiliary electrode) to the device. The additional electrode should only respond to the meteorological conditions and not the target gas, and so provides a reference baseline current. Scientific literature has shown this additional electrode is able to account for steady state effects, but not the rapid changes that cause baseline noise. US20080277290 incorporates a second catalyst on the working electrode surface that responds to an interfering stimulus, such as humidity, in an equal but opposite manner to the gas catalyst. USRE45186 describes an electrochemical gas sensor with humidity compensation, where the humidity compensation is provided by a reservoir of water that keeps the humidity of the membrane at 100%. EP2214008 describes a sensor housing with multilayer walls. The walls have different water vapour transport rates, so that water vapor transport from the atmosphere to the sensor electrolyte is reduced. US20030136675 is a design for an oxygen gas sensor. The sensor housing has a vent hole that brings air from the outside to the sensor electrolyte. A water or oil repellent filter can be inserted into the vent hole to prevent water or oil reaching the sensor. In WO2015123176 a top cap assembly for an electrochemical sensor is described. This is for use with a capillary controlled gas sensor. The cap comprises a capillary controlled gas flow path, a bulk flow membrane, and a raised boss surrounded by a moat. The purpose of the moat is to collect condensation to prevent blockage of gas flow through the capillary. The bulk flow membrane minimises the sensors exposure to rapid changes in external pressure by providing a high resistance to forced bulk flow. Fans have also been used to mitigate changes in external pressure and wind speed in several scientific publications.

Needs exist for improved electrochemical gas sensor with means for providing a baseline of a gas sensor.

### SUMMARY OF THE INVENTION

The current invention describes several improvements to electrochemical gas sensor designs that increase the reliability of the output signal of an electrochemical gas sensor particularly when used for the measurement of gases in outdoor air.

This invention describes improvements to electrochemical gas sensors that improve their reliability and accuracy in ambient air measurement by reducing the effects of rapid changes in meteorological conditions.

### BRIEF DESCRIPTION OF THE DRAWINGS

FIG. 1 shows an electrochemical gas sensor with a housing, a gas inlet, a cap-holding membrane in front of a gas sensor and a fabric membrane.

FIG. 2 shows an electrochemical gas sensor with a housing, an inlet and a cap covering the inlet for measuring a baseline gas sensor.

### DETAILED DESCRIPTION

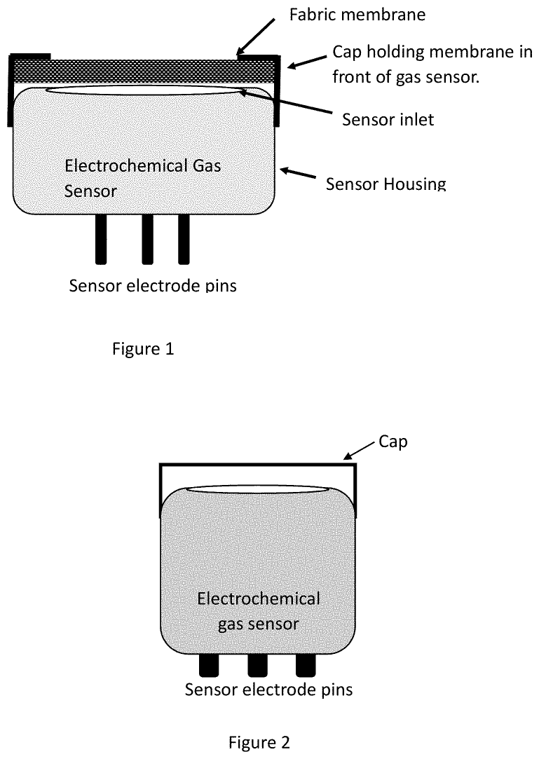

Example 1: A porous fabric membrane positioned in front of the gas inlet of an electrochemical gas sensor (FIG. 1) such as Alphasense NO2 AF43 or Membrapor O3M5 was shown to eliminate the sensors response to changes in humidity but surprisingly did not decrease the sensors response to the target gas. Fabrics composed of natural fibres such as those based on keratin (animal wools, silks), cellulose (cotton, paper) and linen (flax based) as well as manmade viscose and blends of these materials were shown to be effective at reducing rapid humidity response but not appreciably affecting the sensor response to the target gas. Fabrics such as wool and cotton that absorb heat when water is released (water desorption is endothermic) and release heat when water is absorbed (absorption is exothermic) were found to also reduce the electrochemical sensors response to changes in ambient temperature. It seems that the porous fabric membrane buffered changes in the temperature and humidity of the air which contacted the working electrode without significantly decreasing the gas being detected. Placement of the porous fabric membrance is illustrated in Error! Reference source not found.1.

FIG. 1 shows three electrode electrochemical gas sensor enclosed in a sensor housing with a fabric membrane buffering changes in meteorological conditions

Example 2: A cylindrical cap without an orifice at the end is placed over the electrochemical gas sensor and left for a period of time. This process results in the target gas being electrochemically consumed by the sensor until the resultant concentration in the volume contained within the cap falls below the detection limit of the sensor. At this time the sensor baseline current is equivalent to zero concentration. This process of capping the sensor and then waiting for the baseline current to stabilise can be used to calibrate the zero baseline current of the sensor.

FIG. 2 shows a cap without an orifice used to measure baseline current of the sensor.

While the invention has been described with reference to specific embodiments, modifications and variations of the invention may be constructed without departing from the scope of the invention.

We claim:

1. An electrochemical gas sensor with a porous membrane that can absorb and desorb water in response to a change in humidity or dew point or water vapor pressure held in front of the electrochemical gas sensor gas inlet so that the air being sampled by the gas sensor must diffuse through the porous membrane before entering the gas sensor.

2. The electrochemical gas sensor of claim 1 wherein the porous membrance releases heat when water is absorbed and absorbs heat when water is desorbed.

3. The electrochemical gas sensor of claim 1 wherein the porous membrance comprises fibres of one or more of cotton, linen, keratin, wool, silk, and cellulose.

4. The electrochemical gas sensor of claim 1 wherein the filter of claim 3 where the fibres comprise merino wool.

5. The electrochemical gas sensor of claim 1 wherein the porous membrane comprises a manmade fibre including viscose.

6. A method comprising, measuring
Concentrating of gases using an electrochemical gas sensor with a porous membrane inserted in front of the electrochemical gas sensor gas inlet in such a way that the air being sampled by the gas sensor must diffuse through the porous membrane that releases heat when water is absorbed and absorbs heat when water is desorbed before entering the gas sensor.

7. A method comprising calibrating a zero baseline current of an electrochemical gas sensor by covering an inlet of the sensor with a leak tight cap and waiting for a period of time until a current stablises and using resultant stabilized current as the zero baseline current for the sensor calibration.

8. The method of claim 7 wherein the leak tight cap is removed and a cap holding a porous membrane is covering the inlet of the sensor.

9. The method of claim 8 wherein the membrane is a fabric membrane.

10. The method of claim 9 wherein the fabric membrane comprises fibers of one or more cotton, linen, keratin, wool, silk and cellulose.

11. The method of claim 10 wherein the wool comprises merino wool.

12. The method of claim 8 wherein the porous membrane absorbs and disabsorbs water.

13. The method of claim 12 wherein the porous membrane releases heat when water is absorbed and absorbs heat when water is desorbed.
